SEQUENCE LISTING (7 sequences)

SEQ ID NO: 1 - protein, 471 aa, Homo sapiens
  REGION 1..471: note = Description of sequence: hMG53 89A
MSGAPALLQELSAPLALQLFDAPVTAEAGHSFCRACLIKVAGEPGADGTVACPCCQAPSKPQGLSTNQQLGRLVEALAQVPQGHALEHLDPLSVYCEQDRVLVAGVAASLGSHRGHNLLPAAEAHAKLKTQLPQQKMQLQEACMRKEKTVAVLDRQLAEVEETVRQFKGAVGEQLGKMRAFLGALEGSLDREAERVRGEAGVALRRELGTLHSYLDQLRQMERVLEEVGDKPQTEFLMKYCLVTSRLQKILAESPPPARLDIQLPIISDDFKFQVWRKMFRALMPVTEELTFDPSSAHPSLVVSPSGRRVECSEQKAPPAGDDPCQFDKAVAVVAKQQLSEGEHYWEVEVGDKPRWALGVIAADASRRGKLHAVPSQGLWLLGLRDGKILEAHVEAKEPRVLRTPERRPTRIGIYLSFADGVLTFYDASDPDALVPLFTFHERLPGPVYPFFDVCWHDKGKNSQPLLLVGP

SEQ ID NO: 2 - protein, 471 aa, Homo sapiens
  REGION 1..471: note = Description of sequence: hMG53 89C
MSGAPALLQELSCPLCLQLFDAPVTAECGHSFCRACLIKVAGEPGADGTVACPCCQAPSKPQGLSTNQQLGRLVEALAQVPQGHCLEHLDPLSVYCEQDRVLVCGVCASLGSHRGHNLLPAAEAHAKLKTQLPQQKMQLQEACMRKEKTVAVLDRQLAEVEETVRQFKGAVGEQLGKMRAFLGALEGSLDREAERVRGEAGVALRRELGTLHSYLDQLRQMERVLEEVGDKPQTEFLMKYCLVTSRLQKILAESPPPARLDIQLPIISDDFKFQVWRKMFRALMPVTEELTFDPSSAHPSLVVSPSGRRVECSEQKAPPAGDDPCQFDKAVAVVAKQQLSEGEHYWEVEVGDKPRWALGVIAADASRRGKLHAVPSQGLWLLGLRDGKILEAHVEAKEPRVLRTPERRPTRIGIYLSFADGVLTFYDASDPDALVPLFTFHERLPGPVYPFFDVCWHDKGKNSQPLLLVGP

SEQ ID NO: 3 - DNA, 1416 bp, Homo sapiens
  misc_feature 1..1416: note = Description of sequence: nucleic acid of hMG53 89A
  source 1..1416: mol_type = genomic DNA
atgagcggcgctcccgctctgctgcaagaactgtctgctcctctggctctgcagctgttcgatgcccctgtgacagccgaagccggccactctttctgtagagcctgcctgatcaaggtggccggcgaacctggtgctgatggcacagttgcctgtccttgttgtcaggcccctagcaagcctcagggcctgagcacaaatcagcagctgggcagactggtggaagccctggctcaagttcctcagggacacgccctggaacacctggatcctctgtctgtgtactgcgagcaggacagagtgctggtggctggcgttgcagcttctctgggatctcacagaggccacaatctgctgcctgccgccgaagctcacgccaagctgaaaacacagctgccccagcagaagatgcagctccaagaggcctgcatgcggaaagaaaagaccgtggccgtgctggacagacagctggccgaagtggaagaaaccgtgcggcagtttaaaggcgccgtgggagaacagctgggaaagatgagagcctttctgggagccctggaaggcagcctggatagagaagccgagagagttagaggcgaagctggcgtggcactgagaagagagctgggaacactgcacagctacctggaccagctgcggcagatggaacgcgtgctggaagaagtgggcgataagccccagaccgagttcctgatgaagtactgcctggtcaccagcagactgcagaagatcctggccgagtctccacctcctgccagactggatattcagctgcccatcatcagcgacgacttcaagttccaagtgtggcggaagatgttcagagccctgatgcctgtgaccgaggaactgaccttcgatccctctagcgcccatcctagcctggtggtgtctccatctgggagaagagtggaatgcagcgagcagaaagctcctcctgctggcgacgatccctgccagtttgataaggctgtggccgtggtggccaagcagcaactgtctgagggcgagcactactgggaagtcgaagtgggagacaagcctagatgggccctgggagtgattgccgccgatgcctctagaagaggcaagctgcatgccgtgcctagccaaggactgtggctgctgggactgagagatggcaagattctggaagcccacgtggaagccaaagaaccccgggtgctgagaacccctgagagaaggcctaccagaatcggcatctacctgagcttcgccgatggcgtgctgaccttctacgatgccagcgatcctgacgctctggtgcccctgtttaccttccacgagagactgcccggacctgtgtaccccttcttcgatgtgtgctggcacgacaagggcaagaacagccagcctctgctgctcgtgggaccttaa

SEQ ID NO: 4 - DNA, 1416 bp, Homo sapiens
  misc_feature 1..1416: note = Description of sequence: nucleic acid of hMG53 89C
  source 1..1416: mol_type = genomic DNA
atgagcggcgctcccgctctgctgcaagaactgtcttgtcctctgtgcctgcagctgttcgatgcccctgtgacagccgagtgtggccactctttctgcagagcctgcctgattaaggtggccggcgaacctggtgccgatggcacagttgcttgcccttgttgtcaggcccctagcaagcctcagggcctgagcacaaatcagcagctgggcagactggtggaagccctggctcaagttcctcagggacactgcctggaacacctggatcctctgagcgtgtactgcgagcaggacagagtgcttgtgtgtggcgtgtgtgcctctctgggctctcacagaggccacaatttgctgcctgccgccgaagctcacgccaagctgaaaacacagctgccccagcagaagatgcagctccaagaggcctgcatgcggaaagaaaagaccgtggccgtgctggacagacagctggccgaagtggaagaaaccgtgcggcagtttaaaggcgccgtgggagaacagctgggaaagatgagagcctttctgggcgccctggaaggcagcctggatagagaagccgagagagttagaggcgaagctggcgtggccctgagaagagaactgggcacactgcacagctacctggaccagctgcggcagatggaacgcgtgctggaagaagtgggcgataagccccagaccgagttcctgatgaagtactgcctggtcaccagcagactgcagaagatcctggccgagtctccacctcctgccagactggatattcagctgcccatcatcagcgacgacttcaagttccaagtgtggcggaagatgttcagagccctgatgcctgtgaccgaggaactgaccttcgatcccagctctgcccatcctagcctggtggtgtctccatctgggagaagagtggaatgcagcgagcagaaagctcctcctgctggcgacgatccctgccagtttgataaggctgtggccgtggtggccaagcagcaactgtctgagggcgagcactactgggaagtcgaagtgggagacaagcctagatgggccctgggagtgattgccgccgatgcctctagaagaggcaagctgcatgccgtgcctagccaaggactgtggctgctgggactgagagatggcaagattctggaagcccacgtggaagccaaagaaccccgggtgctgagaacccctgagagaaggcctaccagaatcggcatctacctgagcttcgccgatggcgtgctgaccttctacgatgccagcgatcctgacgctctggtgcccctgtttaccttccacgagagactgcccggacctgtgtaccccttcttcgatgtgtgctggcacgataagggcaagaacagccagcctctgctgctcgtgggaccttaa

SEQ ID NO: 5 - protein, 477 aa, Homo sapiens
  REGION 1..477: note = Description of sequence: hMG53
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 6 - protein, 467 aa, Homo sapiens
  REGION 1..467: note = Description of sequence: hMG53 78A
MSGAPGLLELSAPIALQLFEGPVTGEAGHTFCRACLIKAAGEPGADGTVACPSCQAPSKPQGLSTNQQLGRLVEALGQVPQGHALEHLDPLSVYCEQDKVLVAGVAASLGTHRGHNILPAAEAQGKLKSQLPQQKMQLQEACMRKEKTAVVLDRQLAEVEETVRQFKAGVAEQLGKMRAFLGAIEGTIDREAERVAAVVLRVELGTIHSYLDQLRQMERVLEEVGDKPQTEFLMKYCLVSSRLQKILGESPPPARLDIQLPVISDDFKFQVWRKMFRALMPVTQELTFDPTSAHPQLVVSPSARRVECSEQKQPAAGDDPRQFDKAVALVAKQLLSEGEHYWEVEVADKPRWALGVIAGDGSRRGKLHAAPSQGLWLLGLRDGKLIEAHVEAKEPRVLRTPERHPTRIGIYLSFADGVLTFYDASDPDGLILLFTFHERLPGPVYPFFDVCWHDKGKNSQPLILVAP

SEQ ID NO: 7 - protein, 467 aa, Homo sapiens
  REGION 1..467: note = Description of sequence: hMG 78C
MSGAPGLLELSCPICLQLFEGPVTGECGHTFCRACLIKAAGEPGADGTVACPSCQAPSKPQGLSTNQQLGRLVEALGQVPQGHCLEHLDPLSVYCEQDKVLVCGVCASLGTHRGHNILPAAEAQGKLKSQLPQQKMQLQEACMRKEKTAVVLDRQLAEVEETVRQFKAGVAEQLGKMRAFLGAIEGTIDREAERVAAVVLRVELGTIHSYLDQLRQMERVLEEVGDKPQTEFLMKYCLVSSRLQKILGESPPPARLDIQLPVISDDFKFQVWRKMFRALMPVTQELTFDPTSAHPQLVVSPSARRVECSEQKQPAAGDDPRQFDKAVALVAKQLLSEGEHYWEVEVADKPRWALGVIAGDGSRRGKLHAAPSQGLWLLGLRDGKLIEAHVEAKEPRVLRTPERHPTRIGIYLSFADGVLTFYDASDPDGLILLFTFHERLPGPVYPFFDVCWHDKGKNSQPLILVAP

(19) United States
(12) Patent Application Publication
(10) Pub. No.: US 2026/0357223 A1
(43) Pub. Date: Nov. 12, 2026

(54) MODIFIED MG53 POLYPEPTIDES AND METHODS OF USE THEREOF

(71) Applicant: Ohio State Innovation Foundation, Columbus, OH (US)

(72) Inventors: Noah WEISLEDER, Bexley, OH (US); Thomas KWIATKOWSKI, West Chester, PA (US)

(21) Appl. No.: 19/487,685

(22) PCT Filed: May 24, 2024
(86) PCT No.: PCT/US2024/031040

§ 371 Date: Nov. 25, 2025

Related U.S. Application Data
(60) Provisional application No. 63/504,538, filed on May 26, 2023.

(51) Int. Cl. C12N 9/10; A61K 38/00

(52) U.S. Cl. CPC C12N 9/104; A61K 38/00; C12Y 203/02

(57) ABSTRACT

The present disclosure relates compositions and methods to ameliorate diseases or injuries associated with plasma membrane damage and/or repair.

### CROSS-REFERENCE TO RELATED APPLICATION

This application claims priority to, and the benefit of, U.S. Provisional Patent Application No. 63/504,538, filed May 26, 2023, which is incorporated by reference herein in its entirety.

### STATEMENT REGARDING FEDERALLY SPONSORED RESEARCH

This invention was made with Government Support under Grant No. F31-AR080555 awarded by the National Institutes of Health. The Government has certain right in the invention.

### REFERENCE TO SEQUENCE LISTING

The sequence listing submitted on May 24, 2024, as an .XML file entitled “103361-480WO1_ST26” created on May 17, 2024, and having a file size of 14,930 bytes is hereby incorporated by reference pursuant to 37 C.F.R. § 1.52(e)(5).

### FIELD

The present disclosure relates to modified mitsugumin 53 (MG53) polypeptides, compositions, and method of use thereof.

### BACKGROUND

DMD is an X-linked recessive, progressive muscle wasting disorder, the most prevalent form of muscular dystrophy, and the most common fatal genetic disease. DMD leads to compromised skeletal and cardiac muscle structure, decreased muscle and heart function, loss of independence, and death near the second decade of life in affected males. The molecular genetic basis for DMD is disruption of the essential gene dystrophin with hundreds of known mutations in more than 30 classes. Dystrophin mutations compromise sarcolemmal membrane integrity, which leads to excessive injury and ultimate death of muscle fibers. Current treatments have limited or no efficacy on this underlying pathology, thus there is a great unmet need for novel therapies for the pathology associated with DMD as well as other muscular dystrophies and related muscle diseases. Moreover, the cardiac and skeletal muscle damage seen in DMD, other muscular dystrophies and related muscle diseases is similar to that seen in other more common diseases, such as myocardial infarct and resulting heart failure. Novel therapies that can affect these diseases could have a major impact on patient health.

Current cellular and protein therapies under development have limitations. For example, exon-skipping constructs target specific mutations but fail to address most of the >1800 known DMD mutations. Anti-sense oligonucleotides also target specific mutations, but require generation and validation of new oligonucleotides for each patient-specific mutation and may be toxic. Treatment with mini-dystrophin gene constructs are expected to convert DMD pathology into a form that resembles the less severe Becker muscular dystrophy, which is preferable but still has significant pathology. Human adult stem cell therapies may restore lost muscle but face issues such as producing enough transplantable cells and limited cell engraftment.

Given the limitations described above, there is a need to develop therapeutics targeting cardiac and skeletal diseases including, but not limited to DMD, myocardial infarction, and heart failure.

The compositions, and methods disclosed herein address these needs and more.

### SUMMARY

The present disclosure provides modified mitsugumin 53 (MG53) polypeptides and compositions thereof. The present disclosure also provides isolated nucleic acids, expression vectors, and cells expressing the modified mitsugumin 53 (MG53) polypeptides. The present disclosure provides methods of using the modified mitsugumin 53 (MG53) polypeptides for treating and/or preventing cardiac and/or skeletal muscle diseases.

In some aspects, disclosed herein is a modified mitsugumin 53 (MG53) polypeptide comprising one or more mutations that decrease or ablate E3 ligase activity and one or more mutations that reduce or eliminate oligomer formation capacity, wherein the polypeptide retains membrane repair capacity.

In some aspects, disclosed herein is a composition comprising a modified mitsugumin 53 (MG53) polypeptide, wherein the modified MG53 polypeptide comprises one or more mutations that decrease or ablate E3 ligase activity and one or more mutations that reduce or eliminate oligomer formation capacity, wherein the polypeptide retains membrane repair capacity.

In some embodiments, the polypeptide comprises at least 90% sequence identity to SEQ ID NO: 1, or a functional fragment thereof. In some embodiments, the polypeptide comprises SEQ ID NO: 1, or a functional fragment thereof. In some embodiments, the polypeptide comprises at least 90% sequence identity to SEQ ID NO: 2, or a functional fragment thereof. In some embodiments, the polypeptide comprises SEQ ID NO: 2, or a functional fragment thereof.

In some embodiments, the polypeptide comprises at least 90% sequence identity to SEQ ID NO: 1, or a functional fragment thereof, and at least 90% sequence identity to SEQ ID NO: 2, or a functional fragment thereof. In some embodiments, the polypeptide comprises SEQ ID NO: 1, or a functional fragment thereof, and SEQ ID NO: 2, or a functional fragment thereof.

In some embodiments, the membrane repair comprises muscle membrane repair. In some embodiments, the membrane repair is therapeutic for Duchenne muscular dystrophy, Becker muscular dystrophy, limb girdle muscular dystrophies including limb girdle muscular dystrophy 2B/R2, acute lung injury, di-glycan myopathies, inflammatory myopathies, GNE myopathy, Alzheimer's disease, muscle injury, surgery, load bearing exercise recovery, cosmetic membrane repair including skin and bodybuilding membrane repair, acute kidney injury, dermal wounding, peripheral nerve injury, ischemic injury to the heart, liver and/or brain, cardiovascular disease, cardiac ischemia/reperfusion injury, myocardial infarct, hypoxic injury, eye injury, inflammation, or heart failure.

In some embodiments, the composition of any preceding aspect further comprises a pharmaceutically acceptable carrier or excipient. In some embodiments, the composition of any preceding aspect is administered locally or systemically. In some embodiments, the composition is effective in treating or preventing membrane injury.

In some aspect, disclosed herein is an isolated nucleic acid molecule comprising a nucleotide sequence encoding an amino acid sequence comprising at least 90% sequence identity to SEQ ID NO: 1, or a functional fragment thereof, and at least 90% sequence identity to SEQ ID NO: 2, or a functional fragment thereof.

In some embodiments, the nucleotide sequence encodes SEQ ID NO: 1, or a functional fragment thereof, and SEQ ID NO: 2, or a functional fragment thereof. In some embodiments, the nucleotide sequence comprises at least 90% sequence identity to SEQ ID NO: 3, or a variant thereof, and at least 90% sequence identity to SEQ ID NO: 4, or a variation thereof. In some embodiments, the nucleotide sequence comprises SEQ ID NO: 3, or a variant thereof, and SEQ ID NO: 4, or a variant thereof.

In some aspects, disclosed herein is an expression vector comprising the isolated nucleic acid molecule of any preceding aspect.

In some embodiments, the expression vector comprises a bacterial expression vector, a viral vector, and a mammalian expression vector.

In some aspects, disclosed herein is a host cell comprising the isolated nucleic acid molecule of any preceding aspect, the expression vector of preceding aspect, or the polypeptide of any preceding aspect.

In some aspects, disclosed herein is a method of treating or preventing membrane injury in a subject in need thereof, the method comprising administering to the subject, a composition comprising a modified mitsugumin 53 (MG53) polypeptide and a pharmaceutically acceptable excipient, wherein the modified MG53 polypeptide comprises one or more mutations that decrease or ablate E3 ligase activity and one or more mutations that reduce or eliminate oligomer formation capacity, wherein the polypeptide retains membrane repair capacity.

In some embodiments, the membrane injury is muscle and/or skeletal membrane injury. In some embodiments, the method treats or prevents Duchenne muscular dystrophy, Becker muscular dystrophy, limb girdle muscular dystrophies including limb girdle muscular dystrophy 2B/R2, acute lung injury, di-glycan myopathies, inflammatory myopathies, GNE myopathy, Alzheimer's disease, muscle injury, surgery, load bearing exercise recovery, cosmetic membrane repair including skin and bodybuilding membrane repair, acute kidney injury, dermal wounding, peripheral nerve injury, ischemic injury to the heart, liver and/or brain, cardiovascular disease, cardiac ischemia/reperfusion injury, myocardial infarct, hypoxic injury, eye injury, inflammation, or heart failure.

In some embodiments, the pharmaceutically acceptable excipient comprises a liquid, solution, suspension, gel, cream, ointment, implant, explant, slab gel, or coated contact lens. In some embodiments, the composition is administered acutely or chronically. In some embodiments, the composition is administered locally or systematically.

In some embodiments, the composition is administered at least once per day. In some embodiments, the composition is administered daily, weekly, monthly, bimonthly, quarterly, semiannually, annually, or as needed for the subject. In some embodiments, the composition is administered every other day, five times per week, four times per week, three times per week, two times per week, once daily, twice daily, one to four times daily, continuously, or as frequently or infrequently as needed for the subject.

### BRIEF DESCRIPTION OF FIGURES

The accompanying figures, which are incorporated in and constitute a part of this specification, illustrate several aspects described below.

The accompanying figures, which are incorporated in and constitute a part of this specification, illustrate several aspects described below.

FIGS. 1A and 1B show the diagram of MG53 and MyoTRIM proteins constructs. FIG. 1A shows the schematic of original full-length recombinant human MG53 (rhMG53) and two constructs MyoTRIM (or 89A) and 78A with varying levels of homology (89% or 78% respectively) to the original human MG53 protein sequence. Text below indicates the overall changes and purpose of those modifications. FIG. 1B shows the amino acid sequences of the original human MG53 protein and various MyoTRIM constructs.

FIGS. 2A and 2B show the 78A and 78C constructs do not recapitulate MG53 function. FIG. 2A shows the fluorescent imaging of HEK293 cells transfected with either eGFP, eGFP-tagged wild-type MG53, or indicated MG53 variant, 89A (MyoTRIM), 89C, 78A, 78C, with N-terminal eGFP tag. FIG. 2B shows the western blot analysis of cell lysates from A with anti-GFP and anti-MG53 antibodies. Calculated molecular weights: eGFP (~27 kDa), wild-type eGFP-MG53 (81.5 kDa), eGFP-MG53 variants (~80.5 kDa). These results show that the 78A/C constructs do not recapitulate MG53 function while the 89A/C ones do, indicating that specific, non-obvious mutations are necessary to maintain MG53 function.

FIGS. 3A, 3B, and 3C shows the novel MyoTRIM sequence is as effective as MG53 in increasing membrane repair when expressed in cells. HEK293 cells were transfected with either eGFP, eGFP-MG53, or eGFP-MyoTRIM (SEQ ID NO:1). Cell membranes were injured by an IR laser and FM4-64 dye influx was measured. Fluorescence change over time at the injury site is a measurement of membrane repair capacity. FIG. 3A shows the representative images of transfected HEK293 cells before (left column) and at 60 s after injury (right column). White arrow indicates injury site. FIG. 3B shows the averaged traces of FM4-64 dye influx over time. FIG. 3C shows the area under the curve measurements for the traces summarized in FIG. 3B, *p<0.05 by ANOVA.

FIGS. 4A and 4B show that the MyoTRIM (SEQ ID NO:1) can increase membrane repair following mechanical injury to cells by binding PS. FIG. 4A shows a glass bead wounding assay was used to damage N2A cultured cells. The amount of LDH released from these cells following injury decreases comparably for MG53 and MyoTRIM (SEQ ID NO:1), indicating they are equally effective. FIG. 4B shows the recombinant proteins for eGFP (lane 1), rhMG53 (lane 2) and two versions of MyoTRIM (lane 3, (89A, SEQ ID NO:1) and 4, (89C, SEQ ID NO:2)) were pulled down using beads coated with phosphatidylserine (PS). All proteins were tagged with eGFP to assist with identifying transfected cells. The resulting supernatants were blotted for MG53 (top) and eGFP (bottom). These results show that MyoTRIM is as effective as MG53 at binding PS.

FIGS. 5A, 5B, and 5C show that the MyoTRIM protein can increase membrane repair in heart tissue. FIG. 5A shows slices of myocardium tissue from the Bla/J mouse model of muscular dystrophy tested using a laser injury FM4-64 dye exclusion assay. Myocardial tissue slices were treated with recombinant MG53 (MG53) or MyoTRIM (MT) at the indicated concentrations. MT can increase membrane repair in a dose dependent manner to the extent that Bla/J cardiomyocytes can repair as effectively as those from wild type (WT) mice. FIG. 5B shows the analysis of the area under the curve (AUC) from the FM4-64 fluorescence traces in panel A to confirm that MT is more effective at increasing membrane repair than MG53. ANOVA analysis with **p<0.01 vs WT, #p<0.05, ####p<0.001 vs. MG53 [0.01 μM]. FIG. 5C shows the representative images of FM4-64 fluorescence for cardiac slices following laser injury in the presence of MG53 or MT.

FIGS. 6A and 6B show that the MyoTRIM protein can increase membrane repair in human DMD muscle cells. DMD1 myoblast cells transdifferentiated from a DMD patient's fibroblasts were treated with 500 nM of the indicated protein. Cell membranes were injured by confocal laser microscopy and FM4-64 dye influx was measured. Fluorescence change over time at the injury site is a measurement of membrane repair capacity. FIG. 6A shows the images of DMD1 cells before (left column) and at 30 s after injury (right column). White arrow indicates injury site. (FIG. 6B). Area under the curve measurements for the traces of FM4-64 dye influx over time. These results show that MyoTRIM protein can increase membrane repair following laser injury in immortalized DMD human skeletal muscle myoblasts.

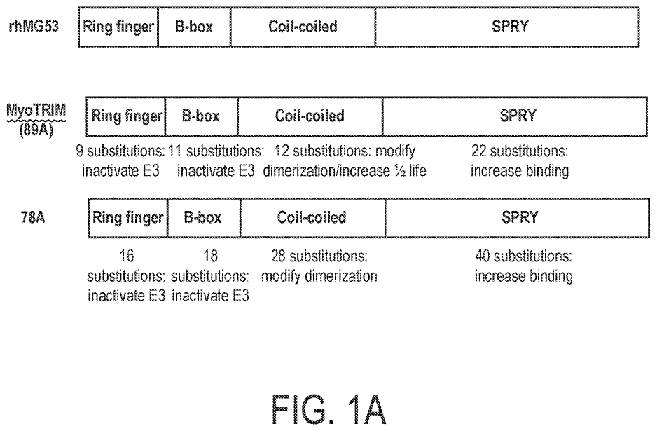

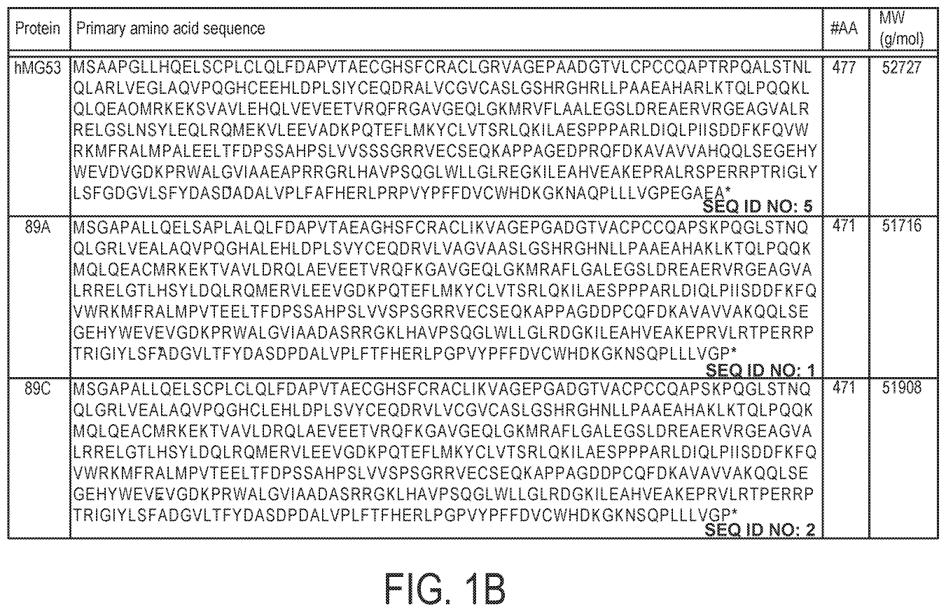

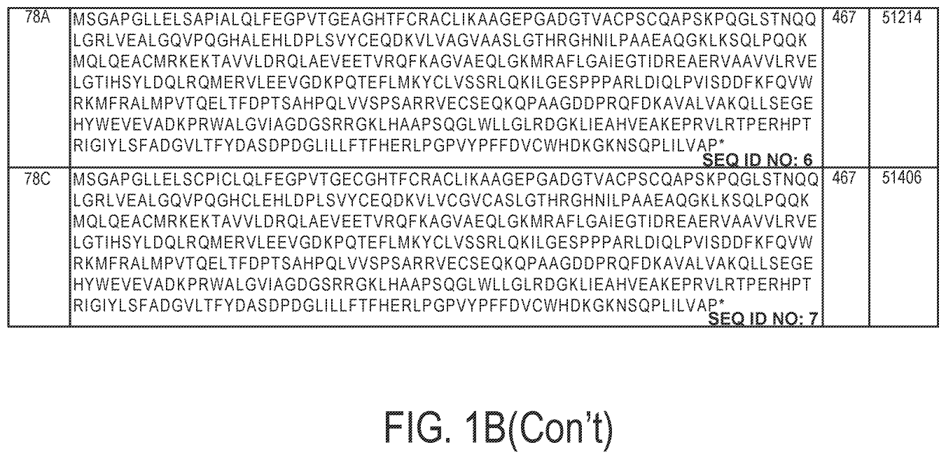

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### TERMINOLOGY

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

Reduction of damage to the structural and functional lipid bilayer of a cell, including maintaining integrity of the plasma membrane to maintain a barrier for cellular contents and facilitate cellular homeostasis.

Plasma membrane damage (“membrane damage”) may be from stressors in the extra- and intra-cellular environment, including chemical, physical, microbial, and immune impacts.

Plasma membrane damage includes, for example, nano ruptures, tears, pore formation, stretch, compression, thermal injury, noise, shear stress, migration, protein aggregates, reactive oxygen species, radiation, phospholipases, amphiphilic molecules, mechanical penetration, trogocytosis, secretion systems, pore-forming toxins, viroporins, complement, perforin, antimicrobials, peroxidation, enzymatic cleavage, and altered fluidity.

As used in the specification and claims, the term “membrane repair” means sealing a wounded/damaged lipid bilayer of a cell via any method, including exocytosis, patching, endocytosis, contraction, plugging, constriction, and scission. Membrane repair may include cellular and extracellular components to facilitate repair, in addition to the polypeptides, nucleic acids, cells, and other aspects of the present invention.

As used in the specification and claims, the singular form “a,” “an,” and “the” include plural references unless the context clearly dictates otherwise. For example, the term “a particle” includes a plurality of particles, including mixtures thereof.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 10% (e.g., within 2% or 1%) of the particular value modified by the term “about.”

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide. The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988).

The term “polynucleotide” refers to a single or double stranded polymer composed of nucleotide monomers.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. As used herein, the terms “reduce”, “decrease”, “ablate”, and “eliminate” can be used interchangeably.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “cell,” “cell line” and “cell culture” include progeny. It is also understood that all progenies may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts. “Pharmaceutically acceptable carrier” (sometimes referred to as a “carrier”) or a “pharmaceutically acceptable excipient” means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

### Mitsugumin 53 Compounds and Compositions

Disclosed herein are compositions and methods to assist membrane repair, including pathologies and injuries wherein membrane repair is therapeutic. For instance, Duchene's muscle dystrophy (DMD), including DMD independent of mutation type, can be ameliorated by use of the present invention. The present disclosure addresses the underlying DMD pathology, and is synergistic with other therapies that improve outcomes even if they do not cure the underlying disease. Protein therapeutics have significant advantages over molecular and stem cell treatments, including a well-defined regulatory path, simplified manufacturing methods and the ability to target multiple muscle diseases. MG53 protein therapy leverages an endogenous protein present in all people, including DMD patients, to avoid immunogenic responses associated with gene replacement approaches. The proposed therapy targets membrane repair mechanisms associated with downstream disease processes that are common across different DMD subtypes, such as membrane fragility and muscle necrosis. Therefore, it addresses a larger target market than gene-specific approaches now in development.

The present disclosure investigates the MG53 protein therapeutic for DMD that enhances the repair capacity of muscle cell membranes compromised by mutations in the dystrophin/dystroglycan complex. Generating an improved engineered version of MG53 represents a novel therapeutic approach by targeting the plasma membrane repair process that seals membrane disruptions. Plasma membrane repair is a highly conserved mechanism in eukaryotic cells and is critical for normal cellular physiology. Enhancing the repair capacity of injury-prone and leaky membranes of dystrophic muscles should improve disease pathology.

These proteins have improved characteristics while remaining functional in vitro and in vivo. The inventors made mutations in the protein that address three major challenges associated with the wild-type MG53 protein sequence: 1) removed the E3 ligase activity of MG53 that was recently linked to potential metabolic effects; 2) increased MG53 protein solubility by minimizing higher molecular weight oligomer formation by altering the protein-protein interaction capacity of the coiled-coil domain of the protein; 3) created unique proteins.

The present disclosure is complementary to therapies now used and/or in development based on delivery of therapeutic genes, proteins and/or cells, because it targets a fundamental membrane repair pathway that is not directly targeted by any other commercial product. DMD patients benefit from both the current disclosure and micro-dystrophin gene therapy. Thus, the current disclosure represents a first-in-class therapeutic for membrane repair pathologies, including DMD and other forms of muscular dystrophy.

The present disclosure is a novel engineered version of MG53, which has improved properties that produces a fundamentally innovative approach to treating membrane repair malfunction, including DMD. Several of the treatments for muscular dystrophy under development use gene therapy approaches to deliver truncated versions of the respective gene via viral vectors. These other approaches are specific to the mutation and gene type, and development of neutralizing antibodies against the viral vector can limit repeat treatment. Because the current disclosure targets a fundamental muscle cell membrane repair process that is not related to the primary DMD mutations, it is amenable to all forms of muscular dystrophy that result from unstable muscle membranes.

The present disclosure is effective to treat both skeletal and cardiac muscle defects, including those involved with DMD. This is increasingly important as the prolonged lifespans, including DMD patients due to the current therapies leads to more concerns about the cardiomyopathy and other long-term sequelae. The current disclosure treats both these target muscle tissues to further prolong the lives of patients.

The present disclosure provides modified mitsugumin 53 (MG53) polypeptides and compositions thereof. The present disclosure also provides isolated nucleic acids, expression vectors, and cells expressing the modified mitsugumin 53 (MG53) polypeptides. The present disclosure provides methods of using the modified mitsugumin 53 (MG53) polypeptides for treating and/or preventing cardiac and/or skeletal muscle diseases.

In some aspects, disclosed herein is a modified mitsugumin 53 (MG53) polypeptide comprising one or more mutations that decrease or ablate E3 ligase activity and one or more mutations that reduce or eliminate oligomer formation capacity, wherein the polypeptide retains membrane repair capacity.

In some aspects, disclosed herein is a composition comprising a modified mitsugumin 53 (MG53) polypeptide, wherein the modified MG53 polypeptide comprises one or more mutations that decrease or ablate E3 ligase activity and one or more mutations that reduce or eliminate oligomer formation capacity, wherein the polypeptide retains membrane repair capacity.

In some embodiments, the polypeptide comprises at least 70% sequence identity to SEQ ID NO: 1, or a functional fragment thereof. In some embodiments, the polypeptide comprises 70%, 75%, 80%, 85%, 90%, 95%, 99%, or 100% sequence identity to SEQ ID NO: 1, or a functional fragment thereof. In some embodiments, the polypeptide comprises SEQ ID NO: 1, or a functional fragment thereof. In some embodiments, the polypeptide comprises at least 70% sequence identity to SEQ ID NO: 2, or a functional fragment thereof. In some embodiments, the polypeptide comprises 70%, 75%, 80%, 85%, 90%, 95%, 99%, or 100% sequence identity to SEQ ID NO: 2, or a functional fragment thereof. In some embodiments, the polypeptide comprises SEQ ID NO: 2, or a functional fragment thereof.

In some embodiments, the polypeptide comprises at least 70% sequence identity to SEQ ID NO: 1, or a functional fragment thereof, and at least 70% sequence identity to SEQ ID NO: 2, or a functional fragment thereof. In some embodiments, the polypeptide comprises 70%, 75%, 80%, 85%, 90%, 95%, 99%, or 100% sequence identity to SEQ ID NO: 1, or a functional fragment thereof, and 70%, 75%, 80%, 85%, 90%, 95%, 99%, or 100% sequence identity to SEQ ID NO: 2, or a functional fragment thereof. In some embodiments, the polypeptide comprises SEQ ID NO: 1, or a functional fragment thereof, and SEQ ID NO: 2, or a functional fragment thereof.

In some embodiments, the membrane repair comprises muscle membrane repair. In some embodiments, the membrane repair is therapeutic for Duchenne muscular dystrophy, Becker muscular dystrophy, limb girdle muscular dystrophies including limb girdle muscular dystrophy 2B/R2, acute lung injury, di-glycan myopathies, inflammatory myopathies, GNE myopathy, Alzheimer's disease, muscle injury, surgery, load bearing exercise recovery, cosmetic membrane repair including skin and bodybuilding membrane repair, acute kidney injury, dermal wounding, peripheral nerve injury, ischemic injury to the heart, liver and/or brain, cardiovascular disease, cardiac ischemia/reperfusion injury, myocardial infarct, hypoxic injury, eye injury, inflammation, or heart failure.

### Nucleic Acids Encoding Mitsugumin 53

In some aspect, disclosed herein is an isolated nucleic acid molecule comprising a nucleotide sequence encoding an amino acid sequence comprising at least 70% sequence identity to SEQ ID NO: 1, or a functional fragment thereof, and at least 70% sequence identity to SEQ ID NO: 2, or a functional fragment thereof.

In some embodiments, the nucleotide sequence encodes 70%, 75%, 80%, 85%, 90%, 95%, 99%, or 100% sequence identity to SEQ ID NO: 1, or a functional fragment thereof, and 70%, 75%, 80%, 85%, 90%, 95%, 99%, or 100% sequence identity to SEQ ID NO: 2, or a functional fragment thereof. In some embodiments, the nucleotide sequence encodes SEQ ID NO: 1, or a functional fragment thereof, and SEQ ID NO: 2, or a functional fragment thereof. In some embodiments, the nucleotide sequence comprises at least 70% sequence identity to SEQ ID NO: 3, or a variant thereof, and at least 70% sequence identity to SEQ ID NO: 4, or a variation thereof. In some embodiments, the nucleotide sequence comprises 70%, 75%, 80%, 85%, 90%, 95%, 99%, or 100% sequence identity to SEQ ID NO: 3, or a variant thereof, and 70%, 75%, 80%, 85%, 90%, 95%, 99%, or 100% sequence identity to SEQ ID NO: 4, or a variant thereof. In some embodiments, the nucleotide sequence comprises SEQ ID NO: 3, or a variant thereof, and SEQ ID NO: 4, or a variant thereof.

In some aspects, disclosed herein is an expression vector comprising the isolated nucleic acid molecule of any preceding aspect. In some embodiments, the expression vector comprises a bacterial expression vector, a viral vector, and a mammalian expression vector.

The word “vector” or “expression vector” refers to any vehicle that carries a polynucleotide into a cell for the expression of the polynucleotide in the cell. The vector may be, for example, a plasmid, a virus, a phage particle, or a nanoparticle. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation.

As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). In some embodiments, the expression vector encoding a chimeric polypeptide is a naked DNA or is comprised in a nanoparticle (e.g., liposomal vesicle, porous silicon nanoparticle, gold-DNA conjugate particle, polyethyleneimine polymer particle, cationic peptides, etc.).

Non-limiting examples of viral vectors include retroviral vectors, adenoviral vectors, adeno-associated viral (AAV) vectors, large payload viral vectors, and lentiviral vectors.

### Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

### Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

In some aspects, disclosed herein is a host cell comprising the isolated nucleic acid molecule of any preceding aspect, the expression vector of preceding aspect, or the polypeptide of any preceding aspect.

### Methods

In some aspects, disclosed herein is a method of treating or preventing membrane injury in a subject in need thereof, the method comprising administering to the subject, a composition comprising a modified mitsugumin 53 (MG53) polypeptide and a pharmaceutically acceptable excipient, wherein the modified MG53 polypeptide comprises one or more mutations that decrease or ablate E3 ligase activity and one or more mutations that reduce or eliminate oligomer formation capacity, wherein the polypeptide retains membrane repair capacity.

In some embodiments, the membrane injury is muscle and/or skeletal membrane injury. In some embodiments, the method treats or prevents Duchenne muscular dystrophy, Becker muscular dystrophy, limb girdle muscular dystrophies including limb girdle muscular dystrophy 2B/R2, acute lung injury, di-glycan myopathies, inflammatory myopathies, GNE myopathy, Alzheimer's disease, muscle injury, surgery, load bearing exercise recovery, cosmetic membrane repair including skin and bodybuilding membrane repair, acute kidney injury, dermal wounding, peripheral nerve injury, ischemic injury to the heart, liver and/or brain, cardiovascular disease, cardiac ischemia/reperfusion injury, myocardial infarct, hypoxic injury, eye injury, inflammation, or heart failure. In some embodiments, the composition of any preceding aspect is effective in treating or preventing membrane injury.

In some embodiments, the pharmaceutically acceptable excipient comprises a liquid, solution, suspension, gel, cream, ointment, implant, explant, slab gel, or coated contact lens. “Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The composition of any preceding aspect may be administered in such amounts, time, and route deemed necessary in order to achieve the desired result. The exact amount of the composition of any preceding aspect will vary from subject to subject, depending on the species, age, and general condition of the subject, the severity of the cardiac and/or skeletal muscle disease, the particular composition, its mode of administration, its mode of activity, and the like. The composition of any preceding aspect is preferably formulated in dosage unit form for ease of administration and uniformity of dosage. It will be understood, however, that the total daily usage of the composition of any preceding aspect will be decided by the attending physician within the scope of sound medical judgment. The specific therapeutically effective dose level for any particular subject will depend upon a variety of factors including the cardiac and/or skeletal muscle disease being treated and the severity of the symptoms; the activity of the composition employed; the specific composition employed; the age, body weight, general health, sex and diet of the patient; the time of administration, route of administration, and rate of excretion of the specific composition employed; the duration of the treatment; drugs used in combination or coincidental with the specific composition employed; and like factors well known in the medical arts.

The composition of any preceding aspect may be administered by any route. In some embodiments, the composition of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition (e.g., its stability in the environment of the subject's body), the condition of the subject (e.g., whether the subject is able to tolerate the chosen route of administration), etc.

The exact amount of the composition of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the composition of any preceding aspect is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the composition of any preceding aspect is administered daily. In some embodiments, the composition of any preceding aspect is administered every day, every 2 days, every 3 days, every 4 days, every 5 days, every 6 days, every 7 days, or more. In some embodiments, the composition of any preceding aspect is administered every week, every 2 weeks, every 3 weeks, every 4 weeks, or more. In some embodiments, the composition of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the composition of any preceding aspect is administered every year, every 2 years, every 3 years, every 4 years, every 5 years, or more. In some embodiments, composition of any preceding aspect is administered at least once per day. In some embodiments, the composition of any preceding aspect is administered daily, weekly, monthly, bimonthly, quarterly, semiannually, annually, or as needed for the subject. In some embodiments, the composition of any preceding aspect is administered every other day, five times per week, four times per week, three times per week, two times per week, once daily, twice daily, one to four times daily, continuously, or as frequently or infrequently as needed for the subject. In some embodiments, the composition of any preceding aspect is administered acutely or chronically. In some embodiments, the composition of any preceding aspect is administered locally or systematically.

In some embodiments, disclosed herein are methods of preventing, reducing, inhibiting, and/or treating diseases, including diseases or injuries associated that are ameliorated via membrane repair, comprising administering to the subject in need a therapeutically effective amount of the compositions disclosed herein.

In some embodiments, disclosed herein are method of preventing, reducing, inhibiting, and/or treating diseases, including diseases or injuries associated that are ameliorated via membrane repair, comprising administering to the subject in need a therapeutically effective amount of a composition of any preceding aspect.

The disclosed methods can be performed any time prior to and/or after the onset of disease. In some aspects, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of disease; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of disease.

Dosing frequency disclosed herein includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiment, the dosing frequency disclosed herein includes, but is not limited to, at least once a day, twice a day, or three times a day. In some embodiment, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range.

The methods provided are useful in researching, treating, reducing, decreasing, inhibiting, and/or preventing membrane repair-associated pathologies and/or injuries.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

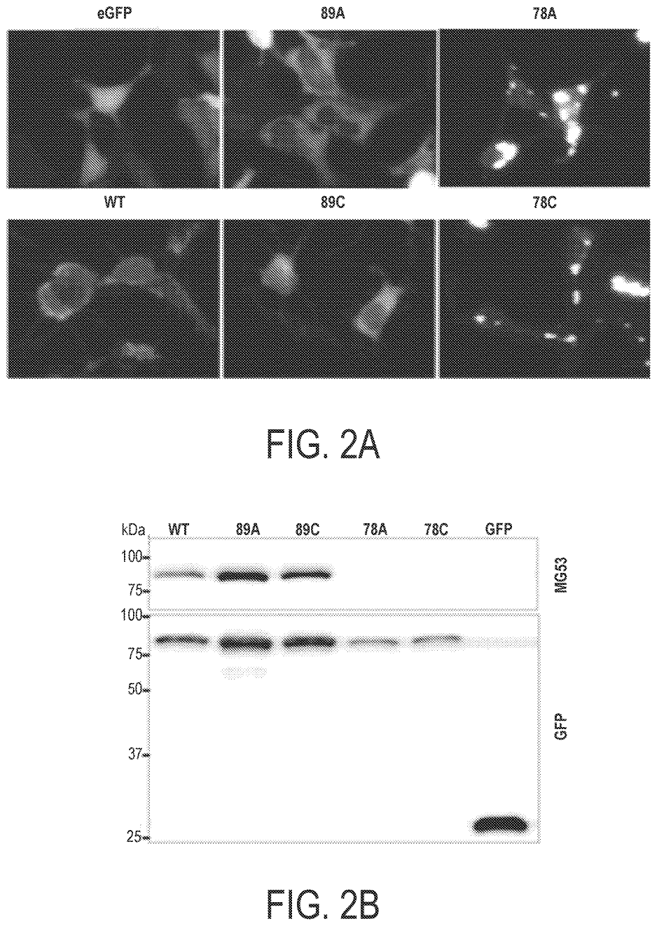

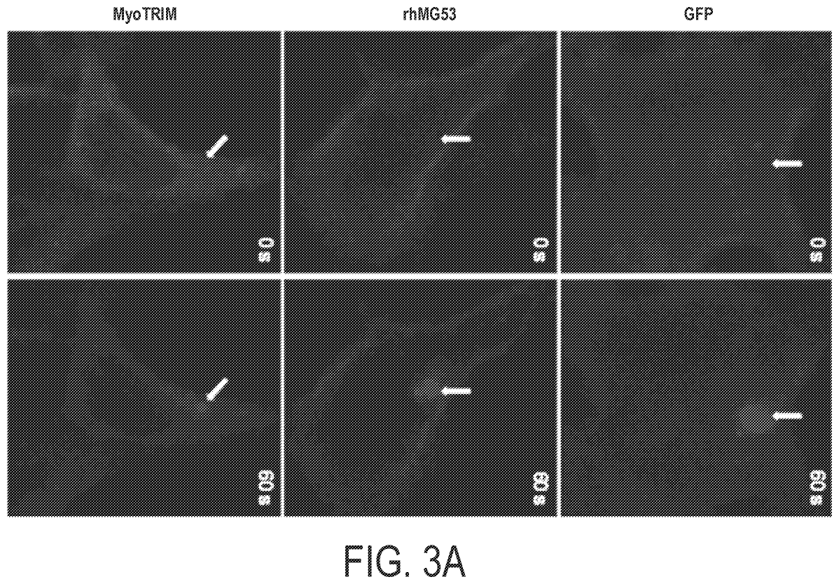

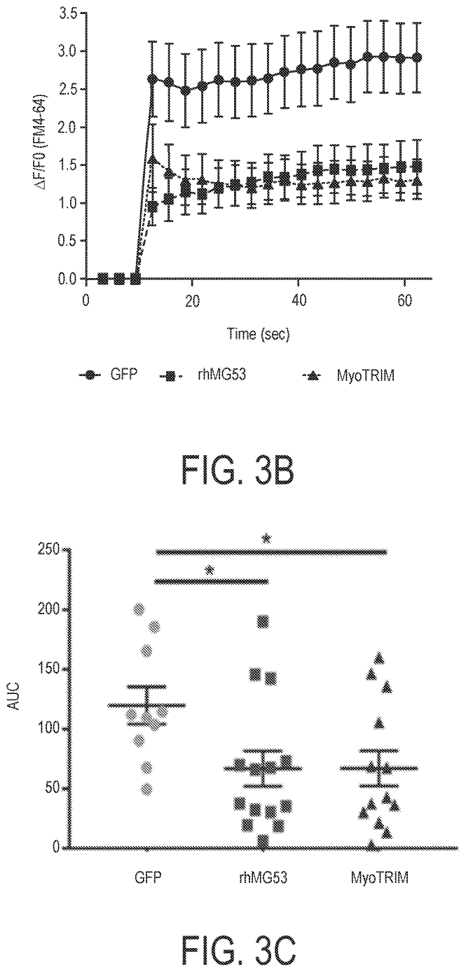

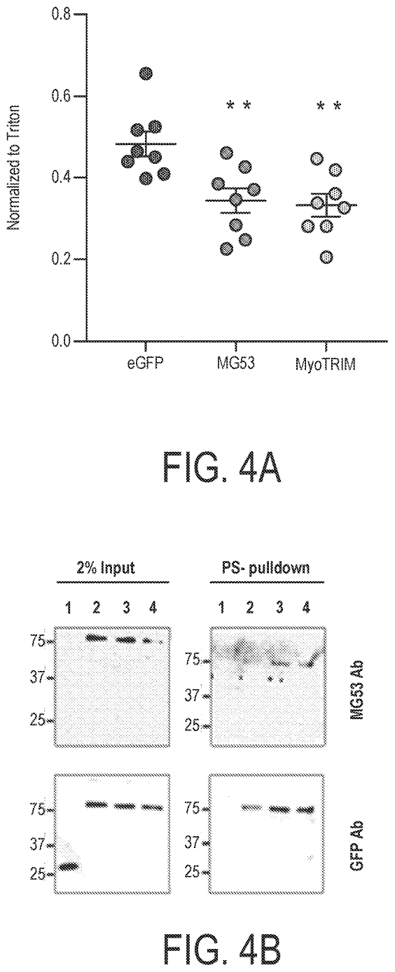

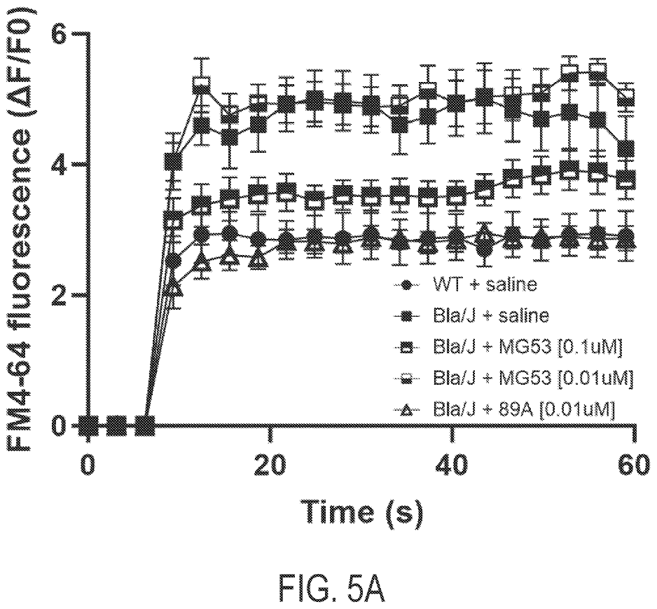

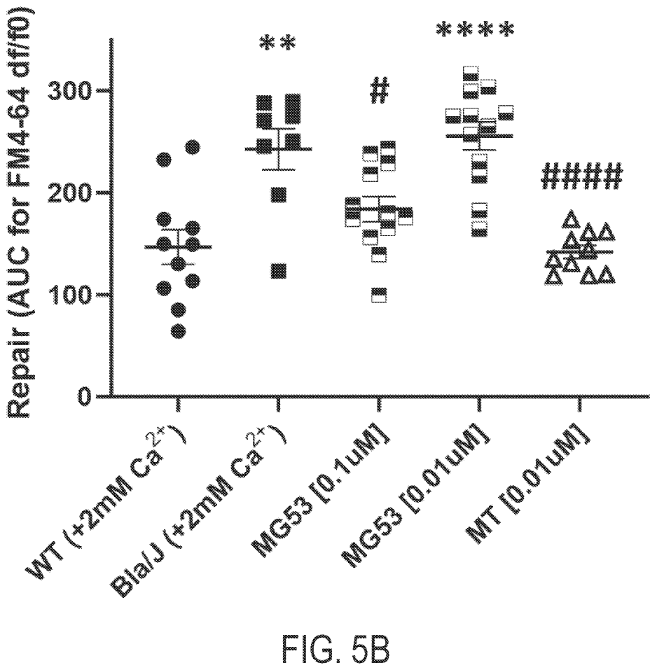

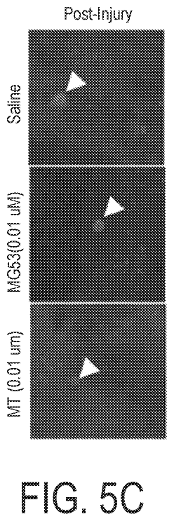

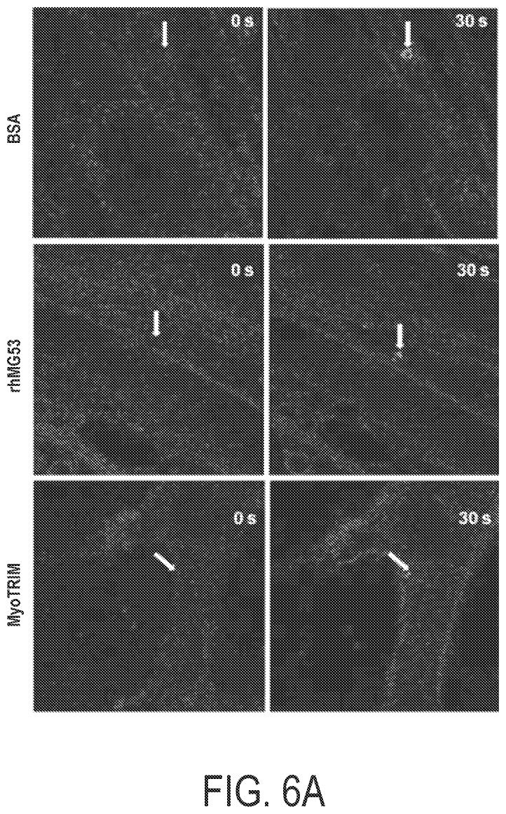

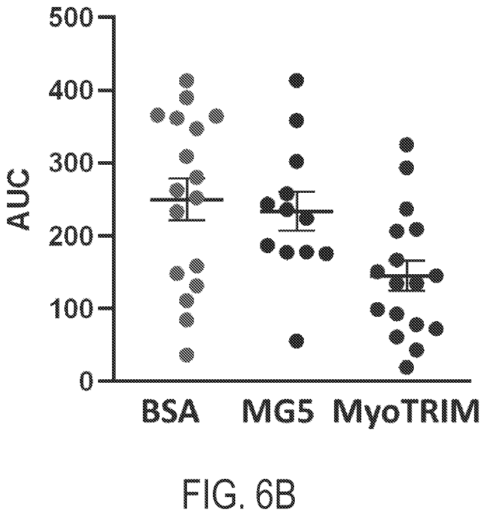

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.
Example 1. HEK293 cells were transfected with either eGFP, eGFP-tagged wild-type MG53, or indicated MG53 variant, 89A, 89C, 78A, 78C, with N-terminal eGFP tag. Fluorescent imaging results are shown in FIG. 2A.
Example 2. Cell lysates from Example 1, were interrogated with anti-GFP and anti-MG53 antibodies. Western blot analysis is shown in FIG. 2B. Calculated molecular weights: eGFP (~27 kDa), wild-type eGFP-MG53 (81.5 kDa), eGFP-MG53 variants (~80.5 kDa).
Example 3. The results of Example 1 and 2 show that the 78A/C constructs do not recapitulate MG53 function while the 89A/C ones do, indicating that specific, non-obvious mutations are necessary to maintain MG53 function. See also Table 1 and Table 2.
Example 4. HEK293 cells were transfected with either eGFP, eGFP-MG53-wt, or eGFP-MyoTRIM (89A). Cell membranes of the transfected cells were injured by an IR laser and FM4-64 dye influx was measured. Fluorescence change over time at the injury site is a measurement of membrane repair capacity. FIG. 3A shows representative images of transfected HEK293 cells before (left column) and at 60 s after injury (right column). White arrow indicates injury site. FIG. 3B shows averaged traces of FM4-64 dye influx over time. FIG. 3C. shows area under the curve measurements for the traces summarized in B, *p<0.05 by ANOVA. These results show Novel MyoTRIM sequence is as effective as MG53 in increasing membrane repair when expressed in cells. See also Table 1 and Table 2.
Example 5. A glass bead wounding assay was used to damage N2A cultured cells. The amount of LDH released from these cells following injury decreases the same amount for MG53 and MyoTRIM (89A), indicating they are equally effective. (FIG. 4A) Recombinant proteins for eGFP (lane 1), MG53 (lane 2) and two versions of MyoTRIM (lane 3, 89A and 4, 89C) were pulled down using beads coated with phosphatidylserine (PS). All proteins were tagged with eGFP to assist with identifying transfected cells. FIG. 4A shows the resulting supernatants were blotted for MG53 (top) and eGFP (bottom). These results show that MyoTRIM is as effective as MG53 at binding PS. See also Table 1 and Table 2.
Example 6. DMD myoblast cells transdifferentiated from DMD patient fibroblasts were treated with 500 nM of the indicated protein. Cell membranes were injured by confocal laser microscopy and FM4-64 dye influx was measured. Fluorescence change over time at the injury site is a measurement of membrane repair capacity. (FIG. 6A) Images of DMD1 cells before (left column) and at 30 s after injury (right column). White arrow indicates injury site. (FIG. 5B). Area under the curve measurements for the traces of FM4-64 dye influx over time, *p<0.05, **p<0.01 by ANOVA. These results show that MyoTRIM protein can increase membrane repair following laser injury in immortalized DMD human skeletal muscle myoblasts. See also Table 1 and Table 2.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Mutational analysis of engineered TRIM72 protein variants. Comprehensive list
of engineered mutations incorporated into TRIM72 protein variants 89A/C
and 78A/C. Colored bars outline canonical protein domains and indicate
mutation location (R = RING, BB = Bbox2, CC = coiled-coil, P = PRY,
and S = SPRY). Mutations that ablate the native E3 ubiquitin ligase
activity are colored red. Mutations are described as either similar or not
similar based on shared physio-chemical properties.

| | Mutation | Type | 89A | 89C | 78A | 78C |
|---|---|---|---|---|---|---|
| R | A3G | Not Similar | X | X | X | X |
| | G6A | Not Similar | X | X | | |
| | H9del | Deletion | X | X | X | X |
| | Q10del | Deletion | | | X | X |
| | C14A | Not Similar | X | | X | |
| | L16I | Similar | | | X | X |
| | C17A | Not Similar | X | | X | |
| | D22E | Similar | | | X | X |
| | A23G | Not Similar | | | X | X |
| | A27G | Not Similar | | | X | X |
| | C29A | Not Similar | X | | X | |
| | S32T | Similar | | | X | X |
| | G39I | Not Similar | X | X | X | X |
| | R40K | Similar | X | X | X | X |
| | V41A | Not Similar | | | X | X |
| | A46G | Not Similar | X | X | X | X |
| | L52A | Not Similar | X | X | X | X |
| | C56S | Not Similar | | | X | X |
| BB | T60S | Similar | X | X | | X |
| | R61K | Similar | X | X | X | X |
| | A64G | Not Similar | X | X | X | X |
| | L69Q | Not Similar | X | X | X | X |
| | A72G | Not Similar | X | X | X | X |
| | G77A | Not Similar | X | X | X | X |
| | A79G | Not Similar | | | X | X |
| | C86A | Not Similar | X | | X | |
| | E87L | Not Similar | X | X | X | X |
| | I95V | Similar | X | X | X | X |
| | R101K | Similar | | | X | X |
| | A102V | Not Similar | X | X | X | X |
| | C105A | Not Similar | X | | X | |
| | C108A | Not Similar | X | | X | |
| | S113T | Similar | | | X | X |
| | R118N | Not Similar | X | X | X | X |
| | L119I | Similar | | | X | X |
| CC | H126Q | Not Similar | | | X | X |
| | A127G | Not Similar | | | X | X |
| | R128K | Similar | X | X | X | X |
| | T131S | Similar | | | X | X |
| | L138M | Similar | X | X | X | X |
| | S150T | Similar | X | X | X | X |
| | V151A | Not Similar | | | X | X |
| | A152V | Not Similar | | | X | X |
| | E155D | Similar | X | X | X | X |
| | H156R | Not Similar | X | X | X | X |
| | V159A | Not Similar | X | X | X | X |
| | R169K | Similar | X | X | X | X |
| | G170A | Not Similar | | | X | X |
| | A171G | Not Similar | | | X | X |
| | G173A | Not Similar | | | X | X |
| | V181A | Not Similar | X | X | X | X |
| | A184G | Not Similar | X | X | X | X |
| | L186I | Similar | | | X | X |
| | S189T | Similar | | | X | X |
| | L190I | Similar | | | X | X |
| | R198del | Deletion | | | X | X |
| | G199del | Deletion | | | X | X |
| | E200del | Deletion | | | X | X |
| | G202A | Not Similar | | | X | X |
| | A204V | Not Similar | | | X | X |
| | R207V | Not Similar | | | X | X |
| | S211T | Similar | X | X | X | X |
| | L212I | Similar | | | X | X |
| | N213H | Similar | X | X | X | X |
| | E217D | Similar | X | X | X | X |
| | K224R | Similar | X | X | X | X |
| | A230G | Not Similar | X | X | X | X |
| | T245S | Similar | | | X | X |
| | A253G | Not Similar | | | X | X |
| P | I267V | Similar | | | X | X |
| | A287V | Not Similar | X | X | X | X |
| | L288T | Not Similar | X | X | X | X |
| | E289Q | Similar | | | X | X |
| | S296T | Similar | | | X | X |
| | S301Q | Not Similar | | X | X | |
| | S306P | Not Similar | X | X | X | X |
| | G308A | Not Similar | | | X | X |
| | A318Q | Not Similar | | | X | X |
| | P320A | Not Similar | | | X | X |
| | E323D | Similar | X | X | X | X |
| | R326C | Not Similar | X | X | | |
| | V334L | Similar | | | X | X |
| | H337K | Not Similar | X | X | X | X |
| | Q339L | Not Similar | | | X | X |
| S | D350E | Similar | X | X | X | X |
| | G352A | Not Similar | | | X | X |
| | A364G | Not Similar | | | X | X |
| | E365D | Similar | X | X | X | X |
| | A366G | Not Similar | | | X | X |
| | P367S | Not Similar | X | X | X | X |
| | R371K | Similar | X | X | X | X |
| | V375A | Not Similar | | | X | X |
| | E387D | Similar | X | X | X | X |
| | I390L | Similar | | | X | X |
| | L391I | Similar | | | X | X |
| | A402V | Not Similar | X | X | X | X |
| | S405T | Similar | X | X | X | X |
| | R409H | Not Similar | | | X | X |
| | L415I | Similar | X | X | X | X |
| | G420A | Not Similar | X | X | X | X |
| | S425T | Similar | X | X | X | X |
| | A432P | Not Similar | X | X | X | X |
| | A434G | Not Similar | | | X | X |
| | V436I | Similar | | | X | X |
| | P437L | Not Similar | | | X | X |
| | A440T | Not Similar | X | X | X | X |
| | R447G | Not Similar | X | X | X | X |
| | A464S | Similar | X | X | X | X |
| | L468I | Similar | | | X | X |
| | G471A | Not Similar | | | X | X |
| | E473del | Deletion | X | X | X | X |
| | G474del | Deletion | X | X | X | X |
| | A475del | Deletion | X | X | X | X |
| | E476del | Deletion | X | X | X | X |
| | A477del | Deletion | X | X | X | X |

TABLE 2
Characterization of engineered TRIM72 protein variants. Summary of characterized biochemical
properties of engineered TRIM72 variants and respective deletion constructs. Deletion constructs
are identified by protein domains, e.g. R = RING, BB = Bbox2, CC = coiled-coil,
P = PRY, and S = SPRY. Qualitative results are presented as text. Quantitative data
is presented as p-value, where * p < 0.05, ** p < 0.01 determined by student T-test or one-way ANOVA.

| | | | | | | |
|---|---|---|---|---|---|---|
| | | | Glass | | | |
| | | | bead | Laser Induced | | |
| | | | Induced | Membrane | Laser Induced | |
| Protein | Subcellular | Western | Membrane | Repair | Membrane Repair | PS |
| Construct | Localization | Blot | Repair | (Endogenous) | (Exogenous) | Binding |

Full Length Protein Constructs

| | | | | | | |
|---|---|---|---|---|---|---|
| 89A-FL | Cytosolic | + | ** | * | p = 0.1867 | +++ |
| 89C-FL | Cytosolic | + | ** | ns | p = 0.5144 | +++ |
| 78A-FL | Puncta | − | | | | |
| 79C-FL | Puncta | − | | | | |

Deletion Protein Constructs

| | | | | |
|---|---|---|---|---|
| 89A-R | Ubiquitous | + | ns | − |
| 89A-R-BB | Cytosolic | + | ns | − |
| 89A-R-BB-CC | Puncta | + | ns | − |
| 89A-CC-P-S | Cytosolic | + | ns | +++ |
| 89A-P-S | Cytosolic | + | ns | + |
| 89A-S | Puncta | + | ** | + |
| 89A-CC | Puncta | + | ** | ++ Nonspecific |
| 89A-R-BB-P-S | Cytosolic | + | | |
| 89A-CC-P | Puncta | + | | |

SEQUENCES

1. SEQ ID NO: 1 - 89A
MSGAPALLQELSAPLALQLFDAPVTAEAGHSFCRACLIKVAGEPGADGTVACPCCQAPS
KPQGLSTNQQLGRLVEALAQVPQGHALEHLDPLSVYCEQDRVLVAGVAASLGSHRGH
NLLPAAEAHAKLKTQLPQQKMQLQEACMRKEKTVAVLDRQLAEVEETVRQFKGAVG
EQLGKMRAFLGALEGSLDREAERVRGEAGVALRRELGTLHSYLDQLRQMERVLEEVG
DKPQTEFLMKYCLVTSRLQKILAESPPPARLDIQLPIISDDFKFQVWRKMFRALMPVTEE
LTFDPSSAHPSLVVSPSGRRVECSEQKAPPAGDDPCQFDKAVAVVAKQQLSEGEHYWE
VEVGDKPRWALGVIAADASRRGKLHAVPSQGLWLLGLRDGKILEAHVEAKEPRVLRTP
ERRPTRIGIYLSFADGVLTFYDASDPDALVPLFTFHERLPGPVYPFFDVCWHDKGKNSQP
LLLVGP
2. SEQ ID NO: 2 - 89C
MSGAPALLQELSCPLCLQLFDAPVTAECGHSFCRACLIKVAGEPGADGTVACPCCQAPS
KPQGLSTNQQLGRLVEALAQVPQGHCLEHLDPLSVYCEQDRVLVCGVCASLGSHRGH
NLLPAAEAHAKLKTQLPQQKMQLQEACMRKEKTVAVLDRQLAEVEETVRQFKGAVG
EQLGKMRAFLGALEGSLDREAERVRGEAGVALRRELGTLHSYLDQLRQMERVLEEVG
DKPQTEFLMKYCLVTSRLQKILAESPPPARLDIQLPIISDDFKFQVWRKMFRALMPVTEE
LTFDPSSAHPSLVVSPSGRRVECSEQKAPPAGDDPCQFDKAVAVVAKQQLSEGEHYWE
VEVGDKPRWALGVIAADASRRGKLHAVPSQGLWLLGLRDGKILEAHVEAKEPRVLRTP
ERRPTRIGIYLSFADGVLTFYDASDPDALVPLFTFHERLPGPVYPFFDVCWHDKGKNSQP
LLLVGP
3. SEQ ID NO: 3 - nucleic acid of SEQ ID NO: 1
ATGAGCGGCGCTCCCGCTCTGCTGCAAGAACTGTCTGCTCCTCTGGCTCTGCAGCTG
TTCGATGCCCCTGTGACAGCCGAAGCCGGCCACTCTTTCTGTAGAGCCTGCCTGATC
AAGGTGGCCGGCGAACCTGGTGCTGATGGCACAGTTGCCTGTCCTTGTTGTCAGGCC
CCTAGCAAGCCTCAGGGCCTGAGCACAAATCAGCAGCTGGGCAGACTGGTGGAAGC
CCTGGCTCAAGTTCCTCAGGGACACGCCCTGGAACACCTGGATCCTCTGTCTGTGTA
CTGCGAGCAGGACAGAGTGCTGGTGGCTGGCGTTGCAGCTTCTCTGGGATCTCACA
GAGGCCACAATCTGCTGCCTGCCGCCGAAGCTCACGCCAAGCTGAAAACACAGCTG
CCCCAGCAGAAGATGCAGCTCCAAGAGGCCTGCATGCGGAAAGAAAAGACCGTGG
CCGTGCTGGACAGACAGCTGGCCGAAGTGGAAGAAACCGTGCGGCAGTTTAAAGG
CGCCGTGGGAGAACAGCTGGGAAAGATGAGAGCCTTTCTGGGAGCCCTGGAAGGC
AGCCTGGATAGAGAAGCCGAGAGAGTTAGAGGCGAAGCTGGCGTGGCACTGAGAA
GAGAGCTGGGAACACTGCACAGCTACCTGGACCAGCTGCGGCAGATGGAACGCGT
GCTGGAAGAAGTGGGCGATAAGCCCCAGACCGAGTTCCTGATGAAGTACTGCCTGG
TCACCAGCAGACTGCAGAAGATCCTGGCCGAGTCTCCACCTCCTGCCAGACTGGAT
ATTCAGCTGCCCATCATCAGCGACGACTTCAAGTTCCAAGTGTGGCGGAAGATGTTC
AGAGCCCTGATGCCTGTGACCGAGGAACTGACCTTCGATCCCTCTAGCGCCCATCCT
AGCCTGGTGGTGTCTCCATCTGGGAGAAGAGTGGAATGCAGCGAGCAGAAAGCTCC
TCCTGCTGGCGACGATCCCTGCCAGTTTGATAAGGCTGTGGCCGTGGTGGCCAAGC
AGCAACTGTCTGAGGGCGAGCACTACTGGGAAGTCGAAGTGGGAGACAAGCCTAG
ATGGGCCCTGGGAGTGATTGCCGCCGATGCCTCTAGAAGAGGCAAGCTGCATGCCG
TGCCTAGCCAAGGACTGTGGCTGCTGGGACTGAGAGATGGCAAGATTCTGGAAGCC
CACGTGGAAGCCAAAGAACCCCGGGTGCTGAGAACCCCTGAGAGAAGGCCTACCA
GAATCGGCATCTACCTGAGCTTCGCCGATGGCGTGCTGACCTTCTACGATGCCAGCG
ATCCTGACGCTCTGGTGCCCCTGTTTACCTTCCACGAGAGACTGCCCGGACCTGTGT
ACCCCTTCTTCGATGTGTGCTGGCACGACAAGGGCAAGAACAGCCAGCCTCTGCTG
CTCGTGGGACCTTAA
4. SEQ ID NO: 4 - nucleic acid of SEQ ID NO: 2
ATGAGCGGCGCTCCCGCTCTGCTGCAAGAACTGTCTTGTCCTCTGTGCCTGCAGCTG
TTCGATGCCCCTGTGACAGCCGAGTGTGGCCACTCTTTCTGCAGAGCCTGCCTGATT
AAGGTGGCCGGCGAACCTGGTGCCGATGGCACAGTTGCTTGCCCTTGTTGTCAGGC
CCCTAGCAAGCCTCAGGGCCTGAGCACAAATCAGCAGCTGGGCAGACTGGTGGAAG
CCCTGGCTCAAGTTCCTCAGGGACACTGCCTGGAACACCTGGATCCTCTGAGCGTGT
ACTGCGAGCAGGACAGAGTGCTTGTGTGTGGCGTGTGTGCCTCTCTGGGCTCTCACA
GAGGCCACAATTTGCTGCCTGCCGCCGAAGCTCACGCCAAGCTGAAAACACAGCTG
CCCCAGCAGAAGATGCAGCTCCAAGAGGCCTGCATGCGGAAAGAAAAGACCGTGG
CCGTGCTGGACAGACAGCTGGCCGAAGTGGAAGAAACCGTGCGGCAGTTTAAAGG
CGCCGTGGGAGAACAGCTGGGAAAGATGAGAGCCTTTCTGGGCGCCCTGGAAGGC
AGCCTGGATAGAGAAGCCGAGAGAGTTAGAGGCGAAGCTGGCGTGGCCCTGAGAA
GAGAACTGGGCACACTGCACAGCTACCTGGACCAGCTGCGGCAGATGGAACGCGTG
CTGGAAGAAGTGGGCGATAAGCCCCAGACCGAGTTCCTGATGAAGTACTGCCTGGT
CACCAGCAGACTGCAGAAGATCCTGGCCGAGTCTCCACCTCCTGCCAGACTGGATA
TTCAGCTGCCCATCATCAGCGACGACTTCAAGTTCCAAGTGTGGCGGAAGATGTTCA
GAGCCCTGATGCCTGTGACCGAGGAACTGACCTTCGATCCCAGCTCTGCCCATCCTA
GCCTGGTGGTGTCTCCATCTGGGAGAAGAGTGGAATGCAGCGAGCAGAAAGCTCCT
CCTGCTGGCGACGATCCCTGCCAGTTTGATAAGGCTGTGGCCGTGGTGGCCAAGCA
GCAACTGTCTGAGGGCGAGCACTACTGGGAAGTCGAAGTGGGAGACAAGCCTAGA
TGGGCCCTGGGAGTGATTGCCGCCGATGCCTCTAGAAGAGGCAAGCTGCATGCCGT
GCCTAGCCAAGGACTGTGGCTGCTGGGACTGAGAGATGGCAAGATTCTGGAAGCCC
ACGTGGAAGCCAAAGAACCCCGGGTGCTGAGAACCCCTGAGAGAAGGCCTACCAG
AATCGGCATCTACCTGAGCTTCGCCGATGGCGTGCTGACCTTCTACGATGCCAGCGA
TCCTGACGCTCTGGTGCCCCTGTTTACCTTCCACGAGAGACTGCCCGGACCTGTGTA
CCCCTTCTTCGATGTGTGCTGGCACGATAAGGGCAAGAACAGCCAGCCTCTGCTGCT
CGTGGGACCTTAA
5. SEQ ID NO: 5 - hMG53
MSAAPGLLHQELSCPLCLQLFDAPVTAECGHSFCRACLGRVAGEPAADGTVLCPCCQA
PTRPQALSTNLQLARLVEGLAQVPQGHCEEHLDPLSIYCEQDRALVCGVCASLGSHRGH
RLLPAAEAHARLKTQLPQQKLQLQEACMRKEKSVAVLEHQLVEVEETVRQFRGAVGE
QLGKMRVFLAALEGSLDREAERVRGEAGVALRRELGSLNSYLEQLRQMEKVLEEVAD
KPQTEFLMKYCLVTSRLQKILAESPPPARLDIQLPIISDDFKFQVWRKMFRALMPALEEL
TFDPSSAHPSLVVSSSGRRVECSEQKAPPAGEDPRQFDKAVAVVAHQQLSEGEHYWEV
DVGDKPRWALGVIAAEAPRRGRLHAVPSQGLWLLGLREGKILEAHVEAKEPRALRSPE
RRPTRIGLYLSFGDGVLSFYDASDADALVPLFAFHERLPRPVYPFFDVCWHDKGKNAQP
LLLVGPEGAEA
6. SEQ ID NO: 6 - 78A
MSGAPGLLELSAPIALQLFEGPVTGEAGHTFCRACLIKAAGEPGADGTVACPSCQAPSK
PQGLSTNQQLGRLVEALGQVPQGHALEHLDPLSVYCEQDKVLVAGVAASLGTHRGHNI
LPAAEAQGKLKSQLPQQKMQLQEACMRKEKTAVVLDRQLAEVEETVRQFKAGVAEQ
LGKMRAFLGAIEGTIDREAERVAAVVLRVELGTIHSYLDQLRQMERVLEEVGDKPQTEF
LMKYCLVSSRLQKILGESPPPARLDIQLPVISDDFKFQVWRKMFRALMPVTQELTFDPTS
AHPQLVVSPSARRVECSEQKQPAAGDDPRQFDKAVALVAKQLLSEGEHYWEVEVADK
PRWALGVIAGDGSRRGKLHAAPSQGLWLLGLRDGKLIEAHVEAKEPRVLRTPERHPTRI
GIYLSFADGVLTFYDASDPDGLILLFTFHERLPGPVYPFFDVCWHDKGKNSQPLILVAP
7. SEQ ID NO: 7 - 78C
MSGAPGLLELSCPICLQLFEGPVTGECGHTFCRACLIKAAGEPGADGTVACPSCQAPSKP
QGLSTNQQLGRLVEALGQVPQGHCLEHLDPLSVYCEQDKVLVCGVCASLGTHRGHNIL
PAAEAQGKLKSQLPQQKMQLQEACMRKEKTAVVLDRQLAEVEETVRQFKAGVAEQL
GKMRAFLGAIEGTIDREAERVAAVVLRVELGTIHSYLDQLRQMERVLEEVGDKPQTEFL
MKYCLVSSRLQKILGESPPPARLDIQLPVISDDFKFQVWRKMFRALMPVTQELTFDPTSA
HPQLVVSPSARRVECSEQKQPAAGDDPRQFDKAVALVAKQLLSEGEHYWEVEVADKP
RWALGVIAGDGSRRGKLHAAPSQGLWLLGLRDGKLIEAHVEAKEPRVLRTPERHPTRI
GIYLSFADGVLTFYDASDPDGLILLFTFHERLPGPVYPFFDVCWHDKGKNSQPLILVAP

1. A modified mitsugumin 53 (MG53) polypeptide comprising one or more mutations that decrease or ablate E3 ligase activity and one or more mutations that reduce or eliminate oligomer formation capacity, wherein the polypeptide retains membrane repair capacity.

2. The polypeptide of claim 1, wherein the polypeptide comprises the amino acid sequence of SEQ ID NO: 1, or a functional fragment thereof, or an amino acid sequence at least 90% identical to SEQ ID NO: 1.

3. The polypeptide of claim 1, wherein the polypeptide comprises the amino acid sequence of SEQ ID NO: 1, or a functional fragment thereof.

4. The polypeptide of claim 1, wherein the polypeptide comprises the amino acid sequence of SEQ ID NO: 2, or a functional fragment thereof, or an amino acid sequence at least 90% identical to SEQ ID NO: 2.

5. The polypeptide of claim 1, wherein the polypeptide comprises the amino acid sequence of SEQ ID NO: 2, or a functional fragment thereof.

6.-7. (canceled)

8. The polypeptide of claim 1, wherein the membrane repair comprises muscle membrane repair.

9. (canceled)

10. An isolated nucleic acid molecule comprising a nucleic acid sequence encoding the modified mitsugumin 53 (MG53) polypeptide of claim 1, wherein the nucleic acid sequence comprises the nucleic acid sequence of SEQ ID NO: 3 or SEQ ID NO: 4, or a nucleic acid sequence at least 90% identical thereto.

11. (canceled)

12. The isolated nucleic acid molecule of claim 10, wherein the nucleic acid sequence comprises the nucleic acid sequence of SEQ ID NO: 3, or a variant thereof.

13. The isolated nucleic acid molecule of claim 10, wherein the nucleic acid sequence comprises the nucleic acid sequence of SEQ ID NO: 4, or a variant thereof.

14. An expression vector comprising the isolated nucleic acid molecule of claim 10.

15. The expression vector of claim 14, wherein the expression vector comprises a bacterial expression vector, a viral vector, or a mammalian expression vector.

16. A host cell comprising the expression vector of claim 14.

17. A pharmaceutical composition comprising a modified mitsugumin 53 (MG53) polypeptide and a pharmaceutically acceptable carrier or excipient, wherein the modified MG53 polypeptide comprises one or more mutations that decrease or ablate E3 ligase activity and one or more mutations that reduce or eliminate oligomer formation capacity, wherein the polypeptide retains membrane repair capacity.

18. The pharmaceutical composition of claim 17, wherein the polypeptide comprises the amino acid sequence of SEQ ID NO: 1, or a functional fragment thereof, or an amino acid sequence at least 90% identical to SEQ ID NO: 1.

19. The pharmaceutical composition of claim 17, wherein the polypeptide comprises the amino acid sequence of SEQ ID NO: 1, or a functional fragment thereof.

20. The pharmaceutical composition of claim 17, wherein the polypeptide comprises the amino acid sequence of SEQ ID NO: 2, or a functional fragment thereof, or an amino acid sequence at least 90% identical to SEQ ID NO: 2.

21. The pharmaceutical composition of claim 17, wherein the polypeptide comprises the amino acid sequence of SEQ ID NO: 2, or a functional fragment thereof.

22. (canceled)

23. (canceled)

24. The pharmaceutical composition of claim 17, wherein the membrane repair comprises plasma membrane repair.

25. (canceled)

26. The pharmaceutical composition of claim 17, wherein the pharmaceutically acceptable carrier or excipient comprises a liquid, solution, suspension, gel, cream, ointment, implant, explant, slab gel, or coated contact lens.

27. (canceled)

28. (canceled)

29. A method of treating or preventing membrane injury in a subject in need thereof, the method comprising administering to the subject a composition comprising a modified mitsugumin 53 (MG53) polypeptide and a pharmaceutically acceptable excipient, wherein the modified MG53 polypeptide comprises one or more mutations that decrease or ablate E3 ligase activity and one or more mutations that reduce or eliminate oligomer formation capacity, and wherein the polypeptide retains membrane repair capacity.

30. The method of claim 29, wherein the membrane injury is muscle and/or skeletal membrane injury.

31. The method of claim 29, wherein the method treats or prevents Duchenne muscular dystrophy, Becker muscular dystrophy, limb girdle muscular dystrophies including limb girdle muscular dystrophy 2B/R2, acute lung injury, di-glycan myopathies, inflammatory myopathies, GNE myopathy, Alzheimer's disease, muscle injury, surgery, load bearing exercise recovery, cosmetic membrane repair including skin and bodybuilding membrane repair, acute kidney injury, dermal wounding, peripheral nerve injury, ischemic injury to the heart, liver and/or brain, cardiovascular disease, cardiac ischemia/reperfusion injury, myocardial infarct, hypoxic injury, eye injury, inflammation, or heart failure.

32. (canceled)

33. The method of claim 29, wherein the composition is administered acutely, chronically, locally, or systemically.

34. (canceled)

35. (canceled)

36. The method of claim 29, wherein the composition is administered at least once daily, twice daily, one to four times daily, continuously, every other day, weekly, two times per week, three times per week, four times per week, five times per week, monthly, bimonthly, quarterly, semiannually, annually, or as frequently or infrequently as needed for the subject.

37. (canceled)
